(19) United States
(12) Patent Application Publication
(10) Pub. No.: US 2018/0353735 A1
(43) Pub. Date: Dec. 13, 2018

(54) SURGICAL DRAIN SECURING DEVICE

(71) Applicant: Timothy J. Frew, Pittsburgh, PA (US)

(72) Inventor: Timothy J. Frew, Pittsburgh, PA (US)

(21) Appl. No.: 16/108,379

(22) Filed: Aug. 22, 2018

Related U.S. Application Data
(63) Continuation-in-part of application No. 15/844,260, filed on Dec. 15, 2017.
(60) Provisional application No. 62/439,152, filed on Dec. 26, 2016.

(51) Int. Cl. A61M 27/00; A61M 25/02

(52) U.S. Cl. CPC A61M 27/00; A61F 5/4404; A61M 2205/0205; A61M 25/02

(57) ABSTRACT

Aspects of the present disclosure are directed to a surgical drain securing device for securing a surgical drain to a body without sutures. The surgical drain securing device alleviates patient discomfort associated with suturing the drain to a patient's skin.

### CROSS-REFERENCE TO RELATED APPLICATIONS

This application is a continuation-in-part of U.S. application Ser. No. 15/844,260, filed 15 Dec. 2017, which claims the benefit of U.S. provisional application No. 62/439,152, filed 26 Dec. 2016, which are hereby incorporated by reference as though fully set forth herein.

### BACKGROUND

### a. Field

The present disclosure generally relates to a device for securing a surgical drain placed in situ within and extending outside of the body.

### b. Background Art

Millions of surgical procedures and operations take place each year. Many surgical procedures create dead space within the body where fluid may collect (i.e., serous, blood, pus, etc.). Surgeons combat fluid build-up by placing drains within the dead space, so that the fluid may escape the body. These drains are secured to the patient via stitching the drain to the skin. This stitch is often a site of discomfort and pain for the patient, causing unnecessary suffering during patient recovery. Moreover, the exit site of the drain is subject to infection.

The foregoing discussion is intended only to illustrate the present field and should not be taken as a disavowal of claim scope.

### BRIEF SUMMARY

The instant disclosure relates to a device for securing a surgical drain to a body without sutures, and thereby alleviates patient discomfort associated with suturing the drain to a patient's skin.

In one embodiment, a surgical drain securing device includes an adhesive section for removably attaching the device to the body, and an aperture attached to or integrally formed with the adhesive section. The surgical drain has a distal end inside the body, a proximal end outside of the body and an intermediate portion between the distal and proximal ends disposed in the aperture of the device.

Another aspect of a preferred embodiment of the present disclosure comprises a device for securing a surgical drain to a body, comprising: a first disk defining an adhesive section for removably attaching the device to the body; and a second disk attached to or integrally formed with the first disk, wherein the second disk has an aperture attached thereto or integrally formed therewith. The surgical drain has a distal end inside the body, a proximal end outside the body and an intermediate portion between the proximal and distal ends disposed in the aperture.

One embodiment of the present disclosure is directed to a device for securing a surgical drain to a body. The device includes a body contacting surface and an aperture. The body contacting surface includes an adhesive layer covering at least a portion of the body contacting surface. The adhesive layer removably attaches the device to the body. The aperture extends through the device and secures an intermediary portion of the surgical drain to the device, and facilitates the flow of fluid from a distal end of the drain within the body to a proximal end of the drain outside the body. In more specific embodiments, the body contacting surface further includes an anti-infection agent layer that covers at least a portion of the body contacting surface. The adhesive layer circumferentially extends around the anti-infection agent layer. The anti-infection agent is absorbed by the body coupled to the device.

Some embodiments of the present disclosure are directed to a surgical drain system for securing a surgical drain to a body. The surgical drain system includes a surgical drain, and a surgical drain securing device. The surgical drain securing device includes an adhesive layer for removably attaching the device to the body, and an aperture that removably couples the surgical drain to the surgical drain securing device. In some specific embodiments of the surgical drain system, the surgical drain includes a distal end that is inserted into the body, and a proximal end that extends out of the surgical drain securing device.

Various aspects of the present disclosure are directed toward methods of installing a surgical drain in a body. In one such embodiment, a method of installing a surgical drain in a body includes the steps of providing the surgical drain, providing a surgical drain securing device, inserting a distal end of the surgical drain into the body through an incision in the body, extending the surgical drain through an aperture in the surgical drain securing device, from a proximal end of the surgical drain to the distal end, coupling the surgical drain securing device to the body via an adhesive layer on the surgical drain securing device, and coupling the surgical drain to the surgical drain securing device via the aperture. In some specific implementations of the method, the method may further include applying an antibiotic composition to the incision via an antibiotic layer applied to a surface of the surgical drain securing device contacting the body.

The foregoing and other aspects, features, details, utilities, and advantages of the present disclosure will be apparent from reading the following description and claims, and from reviewing the accompanying drawings.

### BRIEF DESCRIPTION OF THE DRAWINGS

Various example embodiments may be more completely understood in consideration of the following detailed description in connection with the accompanying drawings, in which:

FIG. 1 is an isometric side view of a surgical drain securing device, consistent with various embodiments of the present disclosure;

FIG. 2 is a top view of the surgical drain securing device of FIG. 1, consistent with various embodiments of the present disclosure;

FIG. 3 is a bottom view of the surgical drain securing device of FIG. 1, consistent with various embodiments of the present disclosure;

FIG. 4A is a side view of a first embodiment of a surgical drain securing device coupled to tissue and securing a surgical drain exiting the tissue, consistent with various embodiments of the present disclosure;

FIG. 4B is a side view of a second embodiment surgical drain securing device coupled to tissue and securing a surgical drain exiting the tissue, consistent with various embodiments of the present disclosure;

FIG. 4C is a cross-sectional side view of a third embodiment of a surgical drain securing device coupled to tissue and securing a surgical drain exiting the tissue, consistent with various embodiments of the present disclosure;

FIG. 5 is an isometric side view of a surgical drain securing device, consistent with various embodiments of the present disclosure;

FIG. 6 is a cross-sectional side view of a surgical drain securing device coupled to a surgical drain, consistent with various embodiments of the present disclosure; and

FIG. 7 is a front view of a patient with a surgical drain securing device securing a surgical drain exiting the patient's chest, consistent with various embodiments of the present disclosure.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION OF EMBODIMENTS

The instant disclosure relates to a device for securing a surgical drain to a body without sutures, and thereby alleviate patient discomfort associated with suturing the drain to a patient's skin, as well as mitigate the risk of infection.

Various aspects of the present disclosure are directed to a surgical drain securing device with two overlapping disks which may be integrally molded together (e.g., one of the overlapping disks may be over-molded to the other disk). A first larger disk may be constructed from a thin pliable plastic, which on a patient contact surface may be impregnated with a strong skin adhesive, or an adhesive layer applied thereto. The first, pliable disk may contour and adhere to variable surface contours of the patient's skin, in proximity to the surgical drain site. An inner disk may comprise a soft silicone, and facilitate passage of the surgical drain through the surgical drain securing device. The inner disk may further include an antibiotic applied to a second patient contact surface (or impregnated with antibiotics). The second patient contact surface may be circumferentially encompassed by the first patient contact surface. The antibiotics may be a specific type of antibiotic which targets skin flora, and decreases the risk of infection associated with the use of such a surgical drain. The inner disk may also have an aperture which facilitates passage of the drain through the surgical drain securing device. The drain may extend through the aperture with a slight interference fit to facilitate securing the drain to the surgical drain securing device. Teeth within the aperture may also facilitate a reduced, dynamic frictional force between the surgical drain securing device and the surgical drain during insertion/extraction of the surgical drain into the surgical drain securing device, while facilitating sufficient static frictional force to prevent the drain from relative movement during use.

A surgical drain securing device may be attached to a patient and reside entirely ex vivo. Once a surgical drain is no longer required, a distal end of the drain may be drawn through the surgical drain securing device, and the adhesive securing the surgical drain securing device to the skin of the patient decoupled therefrom. Alternatively, both the drain and the surgical drain securing device may be removed from the patient simultaneously.

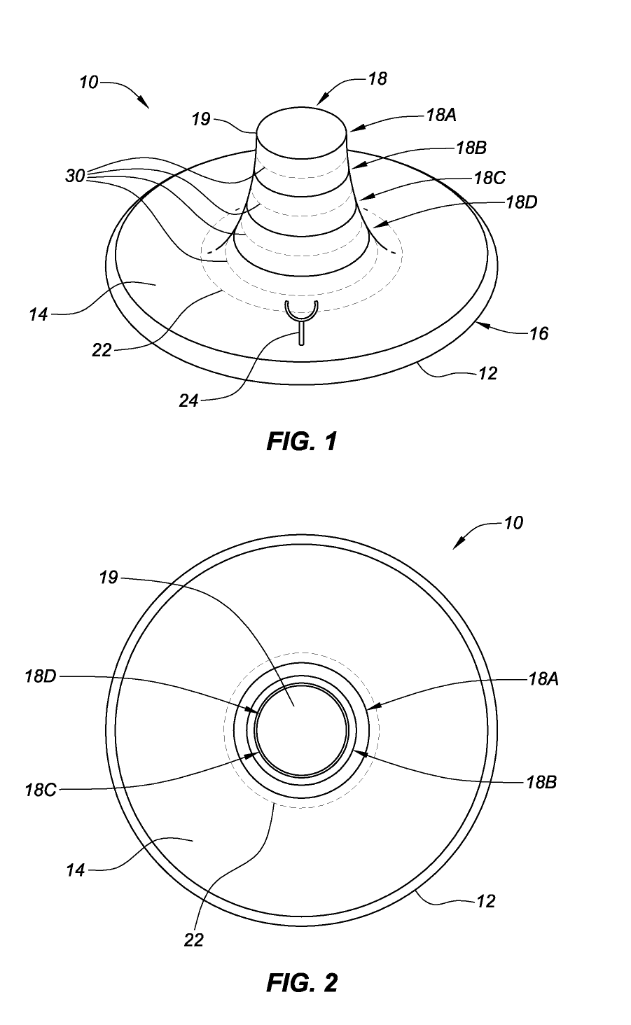

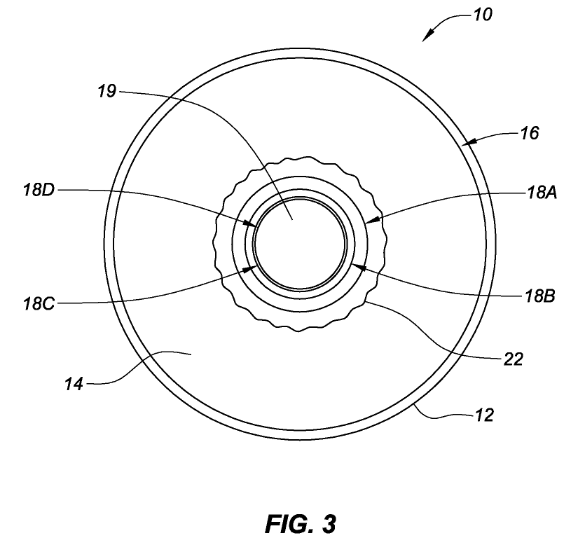

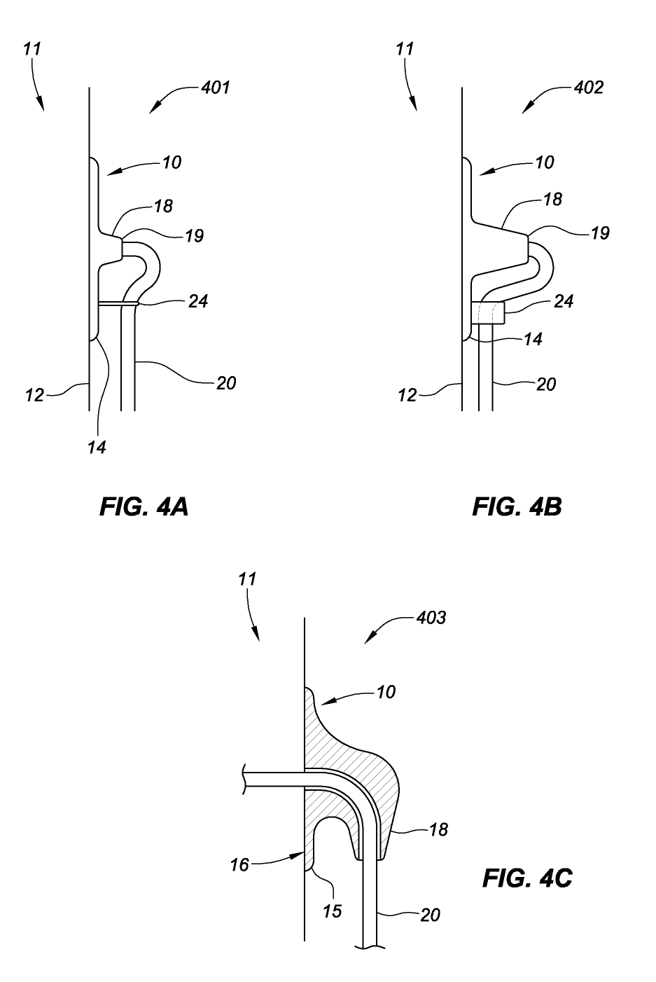

FIG. 1 is an isometric side view of a surgical drain securing device 10, FIG. 2 is a top view of the surgical drain securing device 10 of FIG. 1, FIG. 3 is a bottom view of the surgical drain securing device 10 of FIG. 1, FIG. 4A is a side view of the surgical drain securing device of FIG. 1 coupled to tissue 11 and securing a surgical drain 20 exiting the tissue, consistent with various embodiments of the present disclosure. The resulting assembly 401 facilitates the evacuation of fluid from a surgical site.

As shown in reference to FIGS. 1-4A, surgical drain securing device 10 comprises two overlapping disks 12, 14, which may be molded together (or otherwise coupled to one another) to form the device 10. The first disk 12 may be the larger of the two disks 12, 14, and may be constructed from a thin pliable material, such as silicone, latex, rubber, plastic, similar materials well known to a skilled artisan, or a combination thereof. The underside of disk 12 (that is the tissue contacting side) may be covered, in-whole or in-part, with a strong skin adhesive 16. The first disk 12 may be pliable so that it may contour and adhere to a patient's curvature, no matter where it needs to be affixed to the body (to facilitate a drain exit location).

The second disk 14 may comprise a soft silicone material defining an open-ended tapered cone 18. The tapered cone 18 facilitates passage of a drain tube 20 through device 10 (via an aperture 19) while the outer disk 12 adheres to a patient's body 11. Tapered cone 18 facilitates a fluid-tight seal (via a friction fit) with the drain tube 20. As shown in FIG. 3, an underside of the second disk 14 may have an antibiotic application 22 applied thereto or the second disk is impregnated with antibiotics. The antibiotics may be an ionizable silver, or other similar agents, which target skin flora to decrease the risk of infection. In addition to, or in the alternative, a numbing agent to ease the pain of the incision in which the drain has been inserted may be applied to the second disk or impregnated thereon. As shown in FIGS. 1 and 4A, a hook, clamp or latching device 24 may be attached to device 10 to further secure drain 20 and keep it close to patient's body 11 where it is less likely to catch on objects, such as clothing, etc., and be pulled out of position within the patient's body. Moreover, the latching device may further redirect the drain 20, upon exiting the body, to travel parallel with the body (as shown).

The tapered cone 18, in the present embodiment, defines various inner diameters along its length, from about 0.25 inches near its base 18A, 3/16 inches at a first intermediary location 18B, ⅛ inch at a second intermediary location 18C, to about 3/32 of an inch at distal location 18D. To accommodate surgical drains 20 of different outer diameters, tapered cone 18 may be cut at one of the defined diameters identified by positions 18A-18D, or at any intermediary position between the identified positions along tapered cone 18. Alternatively, tapered cone 18 may comprise a flexible material which may expand and/or contract to facilitate a friction fit over drain tubes 20 of multiple sizes while still providing a fluid-tight seal. However, such an embodiment will result in varying frictional forces between the drain 20 and surgical drain securing device 10.

An inside surface of disk 14, within tapered cone 18, as shown in FIG. 1 may define one or more ribs 30, so that when the device 10 is secured to skin 11 by first disk 12, drain 20 is secured to device 10 via one or more of the ribs 30. This further prevents undesired motion of the drain 20 within the body 11 and seals the exposed portions of the patient's body from an external environment. No part of the device 10 is placed within the patient, and is exclusively applied to the surface of the patient's body (e.g., skin). Once the drain 20 is no longer needed, the adhesive 16 may be decoupled from the skin 11 and the drain 20 removed with device 10 still attached to drain 20. As can be seen, the surgical drain securing device 10 of the present disclosure secures drain 20 in situ inside and outside of body 11.

In reference to FIGS. 1-4A, surgical drain securing device 10 relieves the pain and discomfort often associated with a surgical drain exiting a patient's body 11. As discussed above, first disk 12 may comprise a flexible material. An adhesive 16 is bonded to a bottom surface of the first disk 12, and when coupled with the patient's body secures the device thereto. An ionizable silver ring 22 may also be coupled to an inner diameter of the bottom surface of the first disk 12. The silver ring functions as an antibiotic to help prevent drain site infections. Positions 18A, 18B, 18C, 18D, are pre-defined inner diameters which match the outer diameter sizes of various, common surgical drains. As the drain tubing passes through the corresponding position associated with its outer diameter, the drain will experience a slight interference fit which facilitates securing the drain 20 to the device 10. An aperture 19 facilitates the exit of surgical drain 20 from surgical drain securing device 10. A latching device 24 further secures the surgical drain to the device 10, and prevents the drain from being unintentionally pulled through the device 10.

While various embodiments of the present disclosure show a surgical drain 20 being further coupled to surgical drain securing device 10 via latch device 24 (see, e.g., FIG. 4A), in yet other embodiments the surgical drain may exit open end 19 of the device and hang freely. The device 10 may or may not include a latch device 24.

FIG. 4B is a side view of a second embodiment 402 of a surgical drain securing device 10 coupled to a patient's body 11 and securing a surgical drain 20 exiting the patient, consistent with various embodiments of the present disclosure. Various aspects of the second embodiment of the surgical drain securing device 10 are similar to those described in reference to FIGS. 1-4A. As in FIGS. 1-4A, after exiting an aperture 19 of the device 10, surgical drain 20 is further secured to a latching device 24; however, in FIG. 4B the latching device 24 may be an integral, molded portion of the device 10. The latching device 24 of FIG. 4B facilitates improved atraumatic characteristics of the device 10. Specifically, where a patient may unintentionally contact the device 10, the latching device 24 is less likely to cause trauma to the user. Aspects of the latching device, including a larger surface area, facilitate such atraumatic characteristics. The latching device 24 in FIG. 4B, may form a complete arch through which the drain must be threaded through, or alternatively the latching device 24 may be a semi-circle which may require some deformation of the drain to couple the drain within an inner-diameter of the semi-circle—mitigating decoupling of the latching device and drain during use.

While FIGS. 4A-B are described in reference to a surgical drain securing device 10 with a latching mechanism 24, other aspects of the present disclosure are directed to a device 10 absent such a latching mechanism.

FIG. 4C is a cross-sectional side view of a third embodiment 403 of a surgical drain securing device 10 coupled to a patient's body 11 and securing a surgical drain 20 exiting the body, consistent with various embodiments of the present disclosure. As discussed above in reference to other embodiments, an adhesive 16, on a base 15 of the device 10, facilitates coupling of the device 10 to the body 11. In the present embodiment, the entire device 10 may be injection molded from a plastic such as silicone to achieve desirable flexibility and durability characteristics. Some features of the device 10 may require secondary operations such as machining. Alternatively, the device 10 may be three-dimensionally printed using well known techniques such as stereolithography, fused deposition modeling, digital light processing, among others.

Aspects of the embodiment of FIG. 4C are directed to a surgical drain securing device 10 which internally re-directs a surgical drain 20 extending there through. A surgical drain, as it extends out of the patient's body, is oriented substantially perpendicular to a plane defined by the patient's skin. It has been discovered that by re-directing the surgical drain 20 after it extends out of the patient's body, to extend parallel to the patient's skin, the surgical drain is less prone to snagging and/or being unintentionally withdrawn from the patient's body 11. Accordingly, in the present embodiment a tapered cone 18 of the device 10 includes an approximately 90 degree curve so that the surgical drain 20 exits the device 10 parallel relative to a surface of the patient's body 11. It has been further discovered that the device 10 is less prone to snagging or otherwise being unintentionally decoupled from the patient's body by external forces, and may be less noticeable when worn underneath clothing.

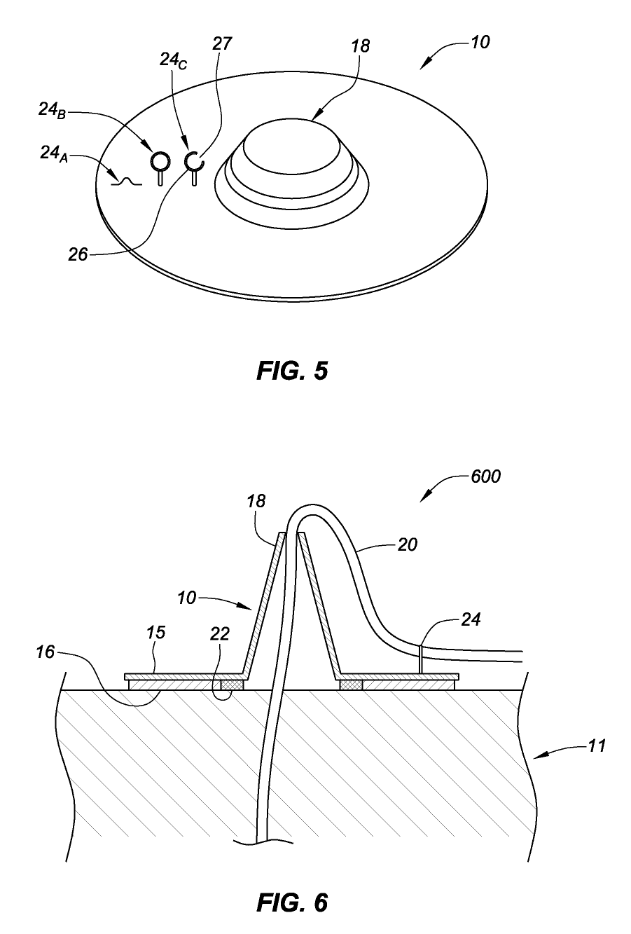

FIG. 5 is an isometric side view of a surgical drain securing device 10, consistent with various embodiments of the present disclosure. In FIG. 5, a number of latching devices 24_{A-C} are shown. The present embodiment is intended to show a few possible implementations of the latching device. In many commercial embodiments, the device 10 may only include a single latching device 24. In various embodiments, the latching device is positioned distal a tapered cone 18, from which the drain extends from, to facilitate a radius of the drain (see, e.g., FIGS. 4A-B) that prevents kinking which would prevent the flow of fluid from the patient.

Latching device 24_{A} is a low profile implementation which minimally extends above a base of the surgical drain securing device 10. In the present embodiment, a surgical drain 20 would be extended through a tapered cone 18 and the latching device. Similarly, a less low-profile implementation of latching device 24_{B} may require the drain 20 to be threaded there through. The eyebolt of latching device 24_{B} may be threaded into the device 10 or otherwise coupled thereto. Latching device 24_{C} is similar to latching device 24_{B}, except that a capture ring 26 does not circumferentially extend about an entire circumference of the drain 20. Accordingly, the drain may be inserted into, and similarly removed, from the capture ring 26 by slightly deforming the drain to fit through an access window 27.

FIG. 6 is a cross-sectional side view of a surgical drain securing device assembly 600 including a surgical drain securing device 10 coupled to a surgical drain 20, consistent with various embodiments of the present disclosure. As shown in FIG. 6, a distal end of the drain 20 extends into a patient's body 11. Many surgical procedures create dead space within a patient's body where fluid may collect. To expel this fluid from the body after an operation, the drain is extended into the dead space to allow for the flow of fluid out of the body as required. In prior art implementations, drains were secured to the patient via sutures. These sutures often caused localized discomfort and pain at the drain/tissue interface. The surgical drain securing device 10 of the present disclosure eliminates the need to suture the drain to the patients' skin and alleviates the patient discomfort associated with suturing the drain to the skin. Instead, as shown in FIG. 6, the drain/tissue interface is circumferentially encompassed by the surgical drain securing device 10, which limits exposure of the interface to bacteria that may otherwise infect the wound. The device 10 is adhered to body 11 via adhesive layer 16 which extends about an outer circumference of a base 15 of the device. An inner circumference of the base 15 may have an antibiotic layer 22 applied thereto. Accordingly, antibiotics from the antibiotic layer 22 may be absorbed by tissue of body 11 in proximity to the drain/tissue interface.

As in the various other embodiments disclosed herein, assembly 600 of FIG. 6 includes a surgical drain 20 which extends out from a surgical drain securing device 10 via a tapered cone 18. The tapered cone 18 also acts as a strain relief for the drain and substantially maintains the position of the distal end of the drain within the body 11. As in many of the other embodiments, a latching mechanism 24 is implemented to re-direct the drain 20 as it extends out of the tapered cone.

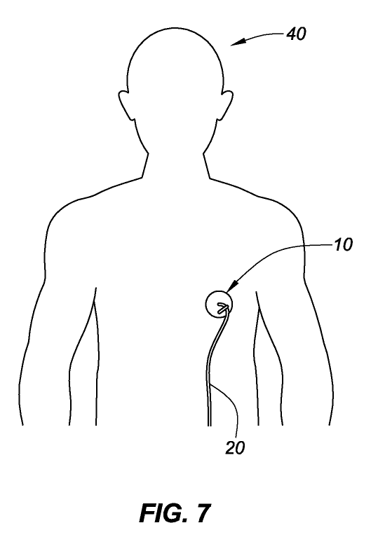

FIG. 7 is a front view of a patient 40 with a surgical drain securing device 10 securing a surgical drain 20 exiting the patient's chest, consistent with various embodiments of the present disclosure. While FIG. 7 shows an application of the surgical drain securing device 10 on a patient's torso, the device may be readily applied to various other locations as a given surgical application dictates. In some embodiments, one or more surgical drain securing devices may be utilized where a large volume of excess fluid is being experienced, or if multiple locations in the patient are experiencing fluid build-up (e.g., operations which require more than one incision site).

In one embodiment of a surgical drain securing device, the device includes an adhesive section for removably attaching the device to a patient's body, and a aperture attached to or integrally formed with the adhesive section. A surgical drain has a distal end extended in vivo, a proximal end ex vivo, and an intermediate portion disposed within the aperture of the surgical drain securing device. In more specific embodiments, the device includes an anti-infection agent applied to or impregnated into the device. The anti-infection agent may comprise an antibiotic or ionizable silver, among other well-known anti-infection compositions. Some embodiments may further include a numbing agent applied to or impregnated into the device which is absorbed by a patient's skin in contact with the device.

A surgical drain securing device may comprise an enclosed hollow body with a aperture extending there through. The aperture may be a tube, a hollow cylinder, a hollow cone, a hollow rectangular tube or a hollow triangular tube. The aperture may further be tapered.

Aspects of the surgical drain securing device may include a clamp (e.g., latching mechanism) for securing a part of the surgical drain after the drain exits the aperture. Moreover, this clamp may be utilized to re-direct the surgical drain. For example, where the surgical drain securing device directs the surgical drain transverse to a plane defined by a patient's skin at the drain site, the clamp may re-direct the drain to extend parallel to the skin plane.

In various embodiments of the present disclosure, a surgical drain securing device may be circular to mitigate the potential for catching on the patient's clothing during use, and to mitigate de-adhering forces on the surgical drain securing device associated with contortion of the patient's skin at the drain site. Similarly, for the above reasons, the adhesive section of the surgical drain securing device may also be generally circular in shape.

Various aspects of the present disclosure are directed to a modifiable surgical drain securing device, where an exterior of the device indicates (via markings, for example) various cutting locations for various size drains. A clinician may shear off a portion of the tapered cone of the device to facilitate use of the device with an over-sized drain diameter. Accordingly, a single surgical drain securing device may be utilized for a range of drain sizes without the need for custom surgical drain securing devices for each of the drain sizes.

Various embodiments of the present disclosure utilize a flexible material to form the surgical drain securing device, which facilitates flexure of the device in response to external forces during use (e.g., the patient's limbs rubbing against the device). In more specific embodiments, the surgical drain securing device may further include structural ribbing (either internal or external) to at least partially stiffen the device.

In some specific implementations of a surgical drain securing device, the device may include an injection port on an exterior side of the device, for injection of antibiotic ointment onto skin in close proximity with the surgical incision. In other specific embodiments, the device may include an antibiotics reservoir tank which facilitates the application of antibiotic ointment onto tissue in close proximity to the surgical incision. The antibiotics reservoir tank may comprise a flexible material such as the rest of the device to facilitate external actuation of the reservoir tank by a user to draw antibiotic ointment from the reservoir tank and into contact with the tissue.

In one embodiment of a surgical drain securing device, the device may comprise a structure that is (largely) collapsible. For example, the device may include an accordion-type manifold which facilitates customizable manipulation of the device by a clinician during installation on a body. For example, the user may manipulate the device so that a tapered cone may include a radius which facilitates an entrance and exit for an aperture extending through the tapered cone having a radial offset of approximately ninety degrees. Similarly, a clinician may extend or contract the tapered cone to achieve a desired depth from the surface of the body.

In one example embodiment of the present disclosure, a device for securing a surgical drain to a body is disclosed. The device includes a first disk defining an adhesive section for removably attaching the device to a patient's body, and a second disk attached to or integrally formed with the first disk. The second disk including an aperture attached thereto or integrally formed therewith. The surgical drain including a distal end positioned in vivo, and a proximal portion positioned ex vivo. An intermediary portion of the drain disposed within the aperture of the device. The first disk may further include an anti-infection agent or numbing agent applied to or impregnated thereon.

Based upon the above discussion and illustrations, those skilled in the art will readily recognize that various modifications and changes may be made to the various embodiments without strictly following the exemplary embodiments and applications illustrated and described herein. For example, the surgical drain securing device may not include a tapered cone, but instead may be formed from one or more of a variety of shapes which facilitate adhesion of the device to tissue and securing of the drain thereto. Such modifications do not depart from the true spirit and scope of various aspects of the invention, including aspects set forth in the claims.

Although several embodiments have been described above with a certain degree of particularity, those skilled in the art could make numerous alterations to the disclosed embodiments without departing from the spirit of the present disclosure. It is intended that all matter contained in the above description or shown in the accompanying drawings shall be interpreted as illustrative only and not limiting. Changes in detail or structure may be made without departing from the present teachings. The foregoing description and following claims are intended to cover all such modifications and variations.

Various embodiments are described herein of various apparatuses, systems, and methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” “an embodiment,” or the like, means that a particular feature, structure, or characteristic described in connection with the embodiment is included in at least one embodiment. Thus, appearances of the phrases “in various embodiments,” “in some embodiments,” “in one embodiment,” “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A device for securing a surgical drain to a body comprising:
a body contacting surface including an adhesive layer covering at least a portion of the body contacting surface, the adhesive layer configured and arranged to removably attach the device to the body; and
an aperture that extends through the device and is configured and arranged to secure an intermediary portion of the surgical drain to the device, and facilitate the flow of fluid from a distal end of the drain within the body to a proximal end of the drain outside the body.

2. The device of claim 1, wherein the body contacting surface further includes an anti-infection agent layer that covers at least a portion of the body contacting surface, the adhesive layer circumferentially extends around the anti-infection agent layer, and wherein the anti-infection agent is configured and arranged to be absorbed by the body coupled to the device.

3. The device of claim 2, wherein the anti-infection agent layer includes an antibiotic or ionizable silver.

4. The device of claim 1, further including a numbing agent layer that covers at least a portion of the body contacting surface, and is configured and arranged to be absorbed by the body coupled to the device.

5. The device of claim 1, wherein the aperture is a tube, a hollow cylinder, a hollow cone, a hollow rectangular tube or a hollow triangular tube.

6. The device of claim 1, further including a tapered cone and the aperture extends through the tapered cone and also includes a tapered profile, the taper of the aperture configured and arranged to facilitate customization of the device to fit surgical drains of varying diameter via removal of a portion of the tapered cone with an aperture diameter less than the diameter of the surgical drain.

7. The device of claim 1, further including a latching mechanism coupled to an outer portion of the device, the latching mechanism configured and arranged to couple the proximal end of the surgical drain to the device.

8. The device of claim 1, wherein the aperture extends non-linearly through the device and is further configured and arranged to bend the intermediary portion of the surgical drain approximately 90 degrees.

9. The device of claim 1, wherein the device is cylindrical, and the adhesive layer extends circumferentially about an outer diameter of the body contacting surface.

10. The device of claim 1, further including one or more ribs disposed within the aperture, the ribs configured and arranged to structurally enforce the aperture.

11. The device of claim 6, wherein the tapered cone includes one or more markings configured and arranged to identify various locations to sever the tapered cone to facilitate coupling of various surgical drains to the aperture.

12. A surgical drain system for securing a surgical drain to a body, comprising:
a surgical drain; and
a surgical drain securing device including
an adhesive layer configured and arranged for removably attaching the device to the body, and
an aperture removably coupling the surgical drain to the surgical drain securing device.

13. The system of claim 12, wherein the surgical drain includes a distal end configured and arranged to be inserted into the body, and a proximal end that extends out of the surgical drain securing device.

14. The system of claim 12, wherein the surgical drain securing device further includes an anti-infection agent layer that circumferentially extends about the aperture on a body contacting surface of the device, and wherein the adhesive layer circumferentially extends about the anti-infection agent on the body contacting surface.

15. The system of claim 12, wherein the surgical drain securing device further includes a numbing agent layer that is applied to the body contacting surface of the device, and wherein the adhesive layer circumferentially extends about the numbing agent layer.

16. A method of installing a surgical drain in a body comprising the steps of:
providing the surgical drain;
providing a surgical drain securing device;
inserting a distal end of the surgical drain into the body through an incision in the body;
extending the surgical drain through an aperture in the surgical drain securing device, from a proximal end of the surgical drain to the distal end;
coupling the surgical drain securing device to the body via an adhesive layer on the surgical drain securing device; and
coupling the surgical drain to the surgical drain securing device via the aperture.

17. The method of claim 16, further including applying an antibiotic composition to the incision via an antibiotic layer applied to a surface of the surgical drain securing device contacting the body.

18. The method of claim 16, further including applying a numbing agent to the incision via a numbing agent layer applied to a surface of the surgical drain securing device contacting the body.

19. The method of claim 16, further including the step of removing a portion of a tapered cone within which the aperture is housed, the extent of the portion removed being dependent upon the diameter of the surgical drain.

20. The method of claim 16, further including the step of redirecting the surgical drain using the aperture of the surgical drain securing device.
